(19) United States
(12) Patent Application Publication
(10) Pub. No.: US 2016/0095539 A1
(43) Pub. Date: Apr. 7, 2016

(54) SMART BAND, BODY BALANCE MEASURING METHOD OF THE SMART BAND AND COMPUTER-READABLE RECORDING MEDIUM COMPRISING PROGRAM FOR PERFORMING THE SAME

(71) Applicant: ZIKTO, Seoul (KR)

(72) Inventors: Kyung Tae KIM, Seoul (KR); Sung Hyun KIM, Seoul (KR); David Hansuk SUH, Seoul (KR)

(21) Appl. No.: 14/519,889

(22) Filed: Oct. 21, 2014

(30) Foreign Application Priority Data
Oct. 2, 2014 (KR) 10-2014-0133171

(51) Int. Cl. A61B 5/11; G01P 3/00; A61B 5/00; G01P 15/14

(52) U.S. Cl. CPC A61B 5/1123; G01P 15/14; G01P 3/00; A61B 5/0002

(57) ABSTRACT

Provided are a smart band, a body balance measuring method of the smart band, and a computer-readable recording medium including a program for performing the same. The smart band includes: a memory that stores a first balance factor of any one of user's left and right arms; a motion sensor that creates motion data by detecting a motion of the other one of the user's left and right arms; and a control unit that extracts a second balance factor of the other one of the user's left and right arms on the basis of the created motion data, calculates an asymmetry index, using the first and second balance factors, and calculates a final score on the basis of the asymmetry index.

### CROSS-REFERENCE TO RELATED APPLICATION

This application claims priority from Korean Patent Application No. 10-2014-0133171 filed on Oct. 2, 2014 in the Korean Intellectual Property Office, and all the benefits accruing therefrom under 35 U.S.C. 119, the contents of which in its entirety are herein incorporated by reference.

### TECHNICAL FIELD

The present invention relates to a smart band, a body balance measuring method of the smart band, and a computer-readable recording medium including a program for performing the same.

### BACKGROUND

A smart band is a wristband capable of searching various services such as diaries, messages, alarms, and stock quotations through wireless communication. Further, users can download data and can set their accounts through a web browser, depending on services.

Recently, there is an increasing need for a healthcare service through smart bands with increasing concern on those smart bands.

### SUMMARY

The present invention has been made in an effort to provide a smart band that provides body balance, that is, asymmetric body shape information by detecting motions of user's arms.

The present invention has also been made in an effort to provide a method of measuring body balance of the smart band that provides body balance, that is, asymmetric body shape information by detecting motions of user's arms.

The present invention has also been made in an effort to provide a computer-readable recording medium including a program for performing the method of measuring body balance of the smart band that provides body balance, that is, asymmetric body shape information by detecting motions of user's arms.

The objects of the present invention are not limited to those described above and other objects may be made apparent to those skilled in the art from the following description

An embodiment of the present invention provides a smart band including: a memory that stores a first balance factor of any one of user's left and right arms; a motion sensor that creates motion data by detecting a motion of the other one of the user's left and right arms; and a control unit that extracts a second balance factor of the other one of the user's left and right arms on the basis of the created motion data, calculates an asymmetry index, using the first and second balance factors, and calculates a final score on the basis of the asymmetry index.

The motion data may contain acceleration or rotation angular velocity of the user's motion.

The control unit may determine a sign of the motion data by checking whether the user's motion is the motion of the user's left arm or right arm.

The sign of motion data for a motion of the user's left arm and the sign of motion data for a motion of the user's right arm may be opposite to each other.

The motion sensor may include an acceleration sensor measuring acceleration of a user's motion and a gyroscope measuring a rotation angular velocity of a user's motion.

The control unit may correct the rotation angular velocity measured by the gyroscope by reflecting a rotation angle measured by the acceleration sensor, extract first to third rotation angular velocity-integral values by integrating the corrected rotation angular velocity, filter noises in the extracted first to third rotation angular velocity-integral values, and create a rotation matrix, using the filtered first to third rotation angular velocity-integral values.

The smart band may further include a filter for filtering noises in the first to third rotation angular velocity-integral values.

The filter may filter noises in the rotation angular velocity measured by the gyroscope before the correcting.

The rotation angle measured by the acceleration sensor may contain a tangent value.

The control unit may calculate linear acceleration by applying the rotation matrix to the acceleration measured by the acceleration sensor, calculates velocity and displacement by integrating the linear acceleration, performs Fourier transform on the third rotation angular velocity-integral value, and extracts the second balance factor on the basis of the velocity, the displacement, and the Fourier transformed-third rotation angular velocity-integral value.

The smart band may further include a display unit that displays the final score, in which directional axes of each of the first and second rotation angular velocity-integral values may cross each other and may be positioned in the same plane as a liquid crystal surface of the display unit, and a directional axis of the third rotation angular velocity-integral value may cross the directional axes of each of the first and second rotation angular velocity-integral values and may be perpendicular to the liquid crystal surface of the display unit.

The control unit may receive the first balance factor from the memory and calculates the asymmetry index on the basis of the difference between the first balance factor and the second balance factor, and each of the first and second balance factors may contain a plurality of sub-balance factors.

The control unit may calculate a spine score, a shoulder score, and a pelvis score on the basis of the asymmetry index and calculates the final score on the basis of the spine score, the shoulder score, and the pelvis score.

Another embodiment of the present invention provides a method of measuring body balance of a smart band which includes: creating motion data by detecting a motion of any one of user's left and right arms; extracting a first balance factor of any one of the user's left and right arms on the basis of the created motion data; calculating an asymmetry index, using the first balance factor and a second balance factor of the other one of the user's left and right arms stored in a memory; and calculating a final score on the basis of the asymmetry index.

The extracting of the first balance factor may include: determining a sign of the motion data by checking whether the user's motion is the motion of the user's left arm or right arm; extracting first to third rotation angular velocity-integral values on the basis of rotation angular velocity data of the motion data with the determined sign; creating a rotation matrix on the basis of the extracted first to third rotation angular velocity-integral values; calculating linear acceleration by applying the rotation matrix to acceleration data of the measured motion data; and extracting the first balance factor on the basis of the linear acceleration and the third rotation angular velocity-integral value.

The extracting of first to third rotation angular velocity-integral values on the basis of rotation angular velocity data of the motion data with the determined sign may include: correcting rotation angular velocity data of the motion data with the determined sign, by reflecting a rotation angle measured by an acceleration sensor; and extracting first to third rotation angular velocity-integral values by integrating the corrected rotation angular velocity data.

The method may further include filtering noises in the rotation angular velocity data, before the correcting of rotation angular velocity data by reflecting a rotation angle measured by an acceleration sensor.

The creating a rotation matrix on the basis of the extracted first to third rotation angular velocity-integral values may include: filtering noises in the extracted first to third rotation angular velocity-integral values; and creating a rotation matrix, using the filtered first to third rotation angular velocity-integral values.

The extracting of the first balance factor on the basis of the linear acceleration and the third rotation angular velocity-integral values may include: calculating velocity and displacement by integrating the linear acceleration; performing Fourier transform on the third rotation angular velocity-integral value; and extracting the second balance factor on the basis of the velocity, the displacement, and the Fourier transformed-third rotation angular velocity-integral value.

The calculating of an asymmetry index may calculate the asymmetry index on the basis of the difference between the first balance factor and the second balance factor.

The method may further include registering the second balance factor of the other one of the user's left and right arms which is compared with the first balance factor, before the creating of motion data by measuring a motion of any one of the user's left and right arms.

The calculating of a final score on the basis of the asymmetry index may include: calculating a spine score, a shoulder score, and a pelvis score on the basis of the asymmetry index; and calculating the final score on the basis of the spine score, the shoulder score, and the pelvis score.

A computer-readable recording medium of the present invention for achieving another embodiment described above includes a program for performing the method of measuring body balance of a smart band.

The details of the present invention are included in the following detailed description and the accompanying drawings.

The effects of the present invention are not limited to the aforementioned effects, and other effects, which are not mentioned above, will be apparently understood by the person skilled in the art from the recitations of the claims.

### BRIEF DESCRIPTION OF THE DRAWINGS

The above and other features and advantages of the present invention will become more apparent by describing in detail embodiments thereof with reference to the attached drawings in which:

FIG. 1 is a diagram illustrating a smart band according to an embodiment of the present invention and a smartphone linked to the smart band;

FIG. 2 is a block diagram illustrating the smart band according to an embodiment of the present invention;

FIG. 3 is a diagram illustrating a user with the smart band of FIG. 2 on his/her wrist; and

FIGS. 4 to 13 are flowcharts illustrating a method of measuring body balance of the smart band according to an embodiment of the present invention.

### DETAILED DESCRIPTION OF THE EMBODIMENTS

Advantages and features of the present invention and methods of accomplishing the same may be understood more readily by reference to the following detailed description of preferred embodiments and the accompanying drawings. The present invention may, however, be embodied in many different forms and should not be construed as being limited to the embodiments set forth herein. Rather, these embodiments are provided so that this disclosure will be thorough and complete and will fully convey the concept of the invention to those skilled in the art, and the present invention will only be defined by the appended claims. Like reference numerals refer to like elements throughout the specification.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that when an element or layer is referred to as being “on”, “connected to” or “coupled to” another element or layer, it can be directly on, connected or coupled to the other element or layer or intervening elements or layers may be present. In contrast, when an element is referred to as being “directly on”, “directly connected to” or “directly coupled to” another element or layer, there are no intervening elements or layers present. As used herein, the term “and/or” includes any and all combinations of one or more of the associated listed items.

It will be understood that, although the terms first, second, and the like. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

Embodiments are described herein with reference to cross-section illustrations that are schematic illustrations of idealized embodiments (and intermediate structures). As such, variations from the shapes of the illustrations as a result, for example, of manufacturing techniques and/or tolerances, are to be expected. Thus, these embodiments should not be construed as limited to the particular shapes of regions illustrated herein but are to include deviations in shapes that result, for example, from manufacturing. For example, an implanted region illustrated as a rectangle will, typically, have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of a device and are not intended to limit the scope of the present invention.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and this specification and will not be interpreted in an idealized or overly formal sense unless expressly so defined herein.

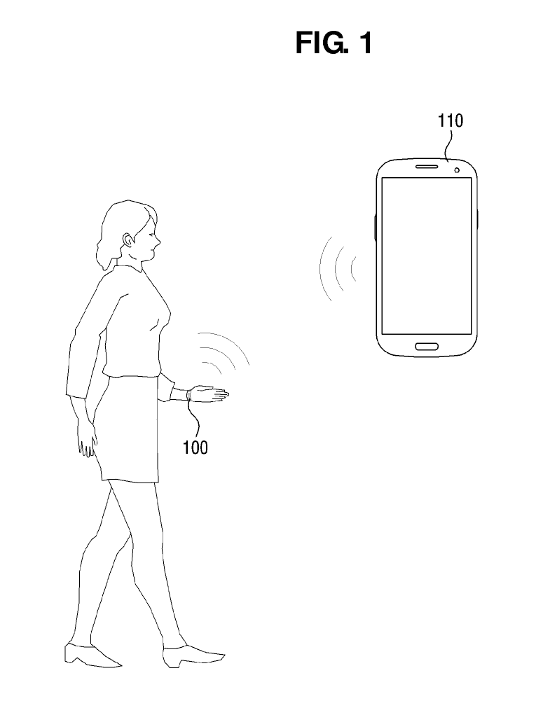

Hereinafter, a smart band according to an embodiment of the present invention and a smartphone linked to the smart band are described with reference to FIG. 1.

FIG. 1 is a diagram illustrating a smart band according to an embodiment of the present invention and a smartphone linked to the smart band.

Referring to FIG. 1, a smart band 100 according to an embodiment of the present invention and a smartphone 110 can communicate with each other, using local communication. The smart band 100, which can be put on a human body (for example, an arm) by a band, has a motion sensor and measures body balance of a user on the basis of a motion of the user with the motion sensor. Accordingly, the user can be provided with his/her body balance (for example, spine, shoulder, pelvis balance) in real time only by walking with the smart band 100 on his/her body without a specific action. The body balance of the user detected through the smart band 100 can be provided to the smartphone 110 too through local communication.

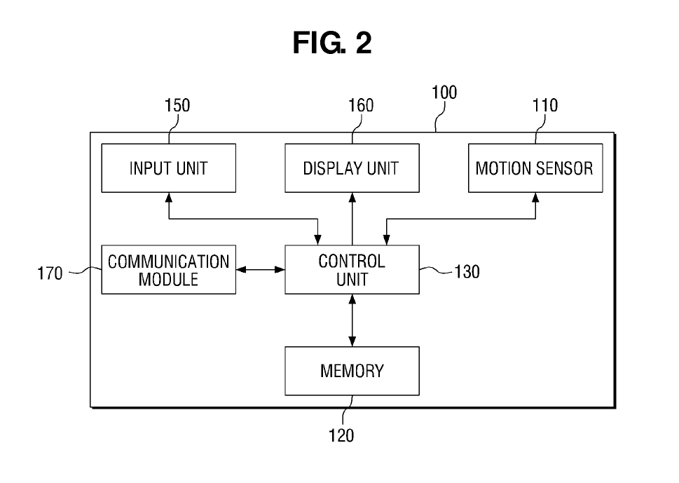

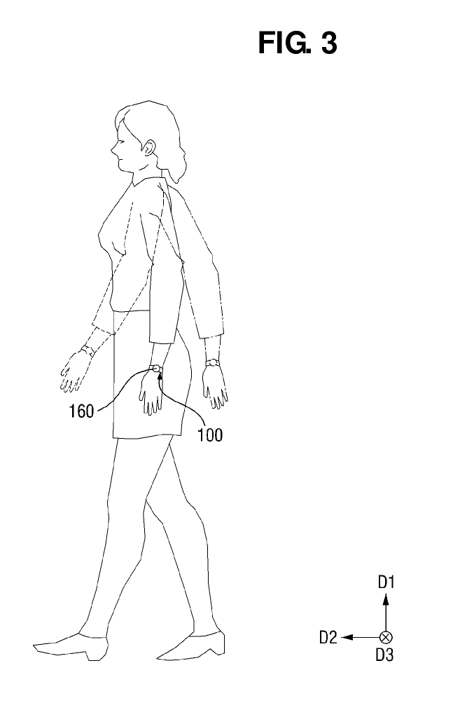

The smart band according to an embodiment of the present invention is described hereafter with reference to FIGS. 2 and 3.

FIG. 2 is a block diagram illustrating the smart band according to an embodiment of the present invention. FIG. 3 is a diagram illustrating a user with the smart band of FIG. 2 on his/her wrist.

Referring to FIG. 2, the smart band 100 according to an embodiment of the present invention includes a motion sensor 110, a memory 120, a control unit 130, an input unit 150, a display unit 160, and a communication module 170.

The motion sensor 110 can create motion data by detecting a motion of a user.

In detail, the motion sensor 110 can create motion data by detecting a motion of any one of the user's left and right arms(for example, the left arm). Further, the motion sensor 110 may include, for example, an acceleration sensor that detects acceleration of a user's motion or a gyroscope that measures rotation angular velocity of a user's motion. Further, the motion sensor 110 is activated periodically or by the control unit 130, so it detects a motion of a user and creates and sends motion data including the detection result to the control unit 130.

The motion data may include acceleration (acceleration data) or a rotation angular velocity (rotation angular velocity data) of a motion of a user, but is not limited thereto.

The memory 120 stores a first balance factor of the other one (for example, the right arm) of the user's left and right arms.

In detail, the memory 120 stores microcodes and reference data of a program for processing and controlling of the control unit 130, temporary data created during execution of various programs, and updatable various data needed to be stored. In particular, the memory 120 can store a registered first balance factor of the other one (for example, the right arm) of the user's left and right arms.

The control unit 130 can detect a motion of a user through the motion sensor 110, create motion data, and find out the body balance of the user on the basis of the motion data.

In detail, the control unit 130 can extract a second balance factor of any one (for example, the left arm) of the user's left and right arms on the basis of the motion data created by the motion sensor 110, calculate an asymmetry index, using the first and second balance factors, and calculate the final score on the basis of the asymmetry index.

The control unit 130 can determine the sign of the motion data by checking whether the user's motion is the motion of the user's left arm or right arm. The sign of the motion data for the motion of the user's left arm and the sign of the motion data for the motion of the user's right arm may be opposite to each other, but the present invention is not limited thereto.

The control unit 130 can correct the rotation angular velocity measured by a gyroscope (not illustrated) by reflecting the rotation angle measured by an acceleration sensor (not illustrated), extract first to third rotation angular velocity-integral values by integrating the corrected rotation angular velocity, filter noises in the extracted first to third rotation angular velocity-integral values, and create a rotation matrix, using the filtered first to third rotation angular velocity-integral values.

The rotation angle measured by the acceleration sensor (not illustrated) may contain, for example, a tangent value, but is not limited thereto.

Referring to FIG. 3, the directional axes D1 and D2 of the first and second rotation angular velocity-integral values may cross each other and may be positioned in the same plane as the liquid crystal surface of the display unit 160, and the directional axis D3 of the third rotation angular velocity-integral value may cross the directional axes D1 and D2 of the first and second rotation angular velocity-integral values and may be perpendicular to the liquid crystal surface of the display unit 160.

The rotation matrix may be expressed, for example, by <Equation 1>

rotationmatrix={cos(yaw(i))*cos(roll(i))cos(yaw(i))*sin(roll(i))*sin(pitch(i))−sin(yaw(i))*cos(pitch(i)) cos(yaw(i))*sin(roll(i))*cos(pitch(i))+sin(yaw(i))*sin(pitch(i));

sin(yaw(i))*cos(roll(i)) sin(yaw(i))*sin(roll(i))*sin(pitch(i))+cos(yaw(i))*cos(pitch(i)) sin(yaw(i))*sin(roll(i))*cos(pitch(i))−cos(yaw(i))*sin(pitch(i));

−sin(roll)(i)) cos(roll(i))*sin(pitch(i))cos(roll)i))*cos(pitch(i))}; <Equation 1>

The first rotation angular velocity-integral value may be pitch(i), the second rotation angular velocity-integral value may be roll(i), and the third rotation angular velocity-integral value may be yaw(i).

Referring to FIG. 2, the control unit 130 may include a filter (not illustrated) that filters noises in the first to third rotation angular velocity-integral values. The filter (not illustrated) can filter noises in the rotation angular velocity measured by the gyroscope before correction and, for example, may be a notch filter, but is not limited thereto.

The control unit 130 can calculate linear acceleration by applying the rotation matrix to the acceleration measured by the acceleration sensor (not illustrated), calculate velocity and displacement by integrating the linear acceleration, perform Fourier transform on the third rotation angular velocity-integral value, and extract the second balance factor on the basis of the velocity, the displacement, and the Fourier transformed-third rotation angular velocity-integral value.

The control unit 130 can receive the first balance factor from the memory 120, calculate the asymmetry index on the basis of the difference between the first balance factor and the second balance factor, calculate a spine score, a shoulder score, and a pelvis score on the basis of the asymmetry index, and calculate the final score on the basis of the spine score, the shoulder score, and the pelvis score.

The first and second balance factors each may contain a plurality of sub-balance factors.

The plurality of sub-balance factors may contain a positive peak (a peak point when a user swings an arm forward) of the third rotation angular velocity-integral value, a negative peak (a peak point when a user swings an arm backward) of the third rotation angular velocity-integral value, the positive and negative peaks in the first direction D1 of FIG. 3 (that is, the positive and negative peak points in the first direction D1 when a user swings an arm), the positive and negative peaks in the second direction D2 of FIG. 3 (that is, the positive and negative peak points in the second direction D2 when a user swings an arm), the positive and negative peaks in the third direction D3 of FIG. 3 (that is, the positive and negative peaks points in the third direction D3 when a user swings an arm), movement time of an arm to the positive peak of the third rotation angular velocity-integral value (movement time of an arm to the peak point when a user swings the arm forward in an attention position), and movement time of an arm to the negative peak of the third rotation angular velocity-integral value (movement time to the peak point when a user swings the arm backward in an attention position), but the present invention is not limited thereto.

The asymmetry index may be calculated, for example, from <Equation 2>.

Asymmetry index=100×(second balance factor−first balance factor)/second balance factor <Equation 2>

The first balance factor may be the balance factor for the motion of the right arm and the second balance factor may be the balance factor for the motion of the left arm, but they are not limited thereto. Further, in <Equation 2> any one sub-balance factor of the second balance factor may be substituted and the corresponding sub-balance factor of the first balance factor may be substituted.

When the first balance factor is the balance factor for the motion of the right arm, the second balance factor is the balance factor for the motion of the left arm, and the asymmetry index is larger than zero, it means that the motion of the left arm is larger.

The control unit 130 can calculate the asymmetry indexes of the sub-balance factors and then calculate the final asymmetry index by summing up the asymmetry indexes.

The final asymmetry index may be calculated, for example, from <Equation 3>.

Final asymmetry index=60+(0.5−(sum of asymmetry indexes))×100 <Equation 3>

The control unit 130, as described above, can calculate the spine score, the shoulder score, and the pelvis score on the basis of an asymmetry index, for example, from the following <Equations 4, 5, and 6>.

Spine score={50+(0.2−(asymmetry index for positive peak of third rotation angular velocity-integral value+asymmetry index for negative peak of third rotation angular velocity-integral value))×200+25+(0.2−(asymmetry index for positive peak in second direction D2 of FIG. 3+asymmetry index for negative peak in second direction D2 of FIG. 3))×100}/1.5 <Equation 4>

Shoulder score=50+(0.2−(asymmetry index for positive peak in first direction D1 of FIG. 3+asymmetry index for negative peak in first direction D1 of FIG. 3))×200 <Equation 5>

Pelvis score={50+(0.2−(asymmetry index for positive peak in third direction D3 of FIG. 3+asymmetry index for negative peak in third direction D3 of FIG. 3))×200+25+(0.2−(asymmetry index for positive peak in second direction D2 of FIG. 3+asymmetry index for negative peak in second direction D2 of FIG. 3))×100}/1.5 <Equation 6>

The final score can be calculated from the spine score, the shoulder score, and the pelvis score with a specific weight.

The input unit 150 can receive information from a user.

In detail, the input unit 150 may be composed of several function keys, and in this case, it sends key input data corresponding to the key pressed by a user to the control unit 130. The functions of the input unit 150 and the display unit 160 may be achieved by a touch screen unit (not illustrated), and in this case, the touch screen unit (not illustrated) is in charge of touch screen input through a touch on a screen by a user and graphic output through a touch screen.

The display unit 160 can display output from the control unit 130.

In detail, the display unit 160 displays state information created in the operation of the smart band 100, a limited number of letters, and a large amount of video images and still images. Further, the display unit 160 may include, for example, a liquid crystal display (LCD).

The communication module 170 can communicate with an electronic device around (for example, a smartphone) in response to signals from the control unit 130.

In detail, the communication module 170 encodes signals from the control unit 130, transmits them to an electronic device around (for example, a smartphone), using local wireless communication such as Bluetooth, ZigBee, infrared, UWB (Ultra Wide Band), WLAN (Wireless LAN), and NFC (Near Field Communication), decodes signals transmitted from the electronic device around through local wireless communication, and then transmits them to the control unit 130.

The smart band 100 according to an embodiment of the present invention can provide body balance, that is, body shape asymmetry information, by detecting the motions of the user's hands through the motion sensor 110 and the control unit 130. Further, the smart band 100 can assist a user to keep good body balance by providing the user with his/her body balance in real time, as described above.

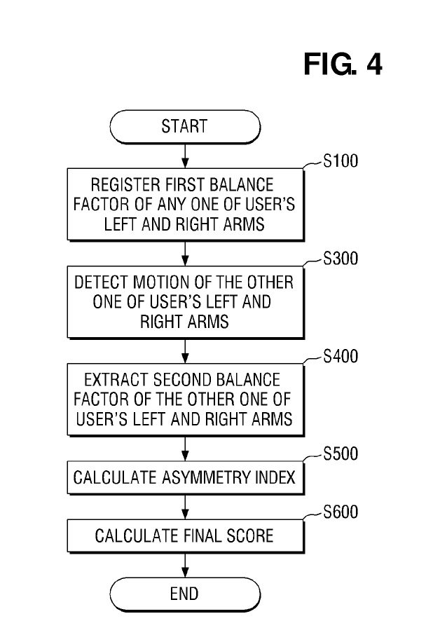

A method of measuring body balance of the smart band is described hereafter with reference to FIGS. 4 to 13.

FIGS. 4 to 13 are flowcharts illustrating a method of measuring body balance of the smart band according to an embodiment of the present invention.

Referring to FIG. 4, the first balance factor of any one (for example, the right arm) of the user's left and right arms is registered first (S100).

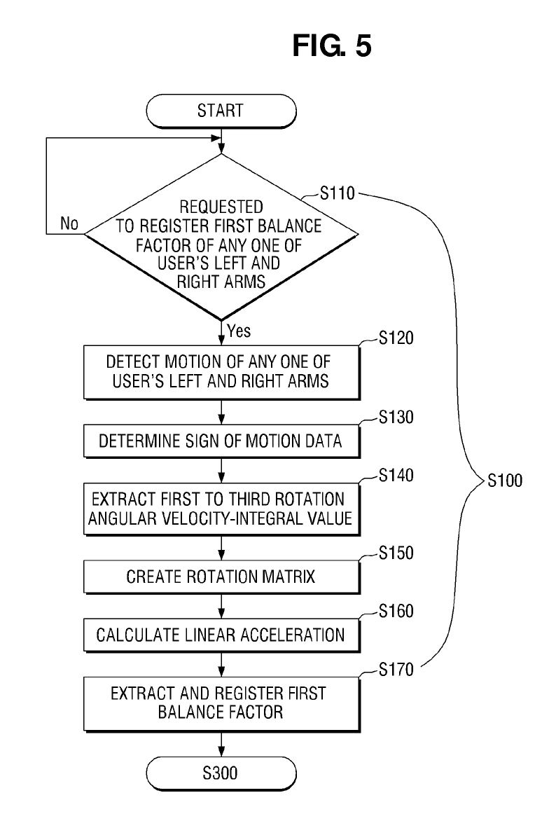

In detail, referring to FIGS. 2 and 5, when requested to register the first balance factor of any one (for example, the right arm) of the user's left and right rams by the user operating a key, the smart band 100 activates the motion sensor 110 and creates motion data by detecting any one (for example, the right arm) of the user's left and right arms for a predetermined time with the motion sensor 110 (S120). For example, when the motion sensor 110 is an acceleration sensor, the smart band 100 measures the acceleration of a user's motion and creates acceleration data, and when the motion sensor 110 is a gyroscope, it measures a rotation angular velocity of a user's motion and creates angular velocity data. The acceleration data contains three axial (x-, y-, and z-axial) acceleration components and the angular velocity data contains three axial angular velocity components.

Next, the sign of the motion data is determined (S130).

In detail, the control unit 130 can determine the sign of the motion data by checking whether the user's motion is the motion of the user's left arm or right arm.

Next, the first to third rotation angular velocity-integral values are extracted (S140).

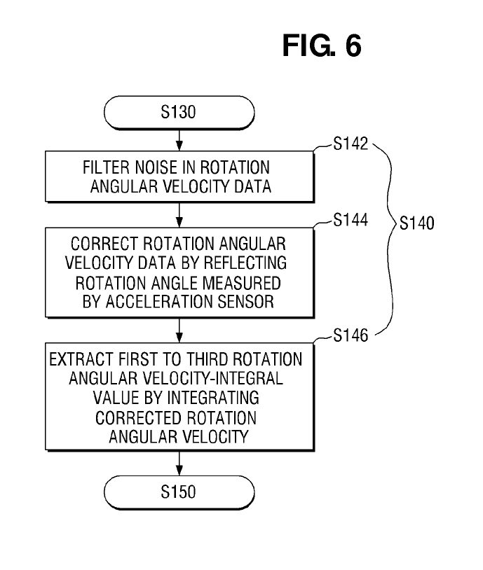

In detail, referring to FIGS. 2 and 6, the control unit 130 can first filter noises in the rotation angular velocity data of the motion data with a sign determined (in which, the filtering may be achieved by a filter (not illustrated) in the control unit 130) (S142), correct the rotation angular velocity data with noises filtered, by reflecting the rotation angle measured by the acceleration sensor (not illustrated) (S144), and extract the first to third rotation angular velocity-integral values by integrating the corrected rotation angular velocity (S146).

Referring to FIGS. 2 and 5 again, a rotation matrix is created (S150).

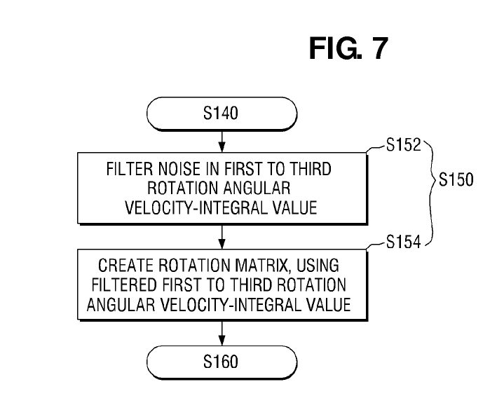

In detail, referring to FIGS. 2 and 7, the control unit 130 can filter noises in the extracted first to third rotation angular velocity-integral values (in which, the filtering may be achieved by a filter (not illustrated) in the control unit 130) (S152) and create a rotation matrix, using the filtered first to third rotation angular velocity-integral values (S154).

Referring to FIGS. 2 and 5 again, linear acceleration is calculated (S160).

In detail, the control unit 130 can calculate the linear acceleration by applying the rotation matrix to the acceleration data of the motion data measured by the motion sensor 110.

Next, the first balance factor is extracted and registered (S170).

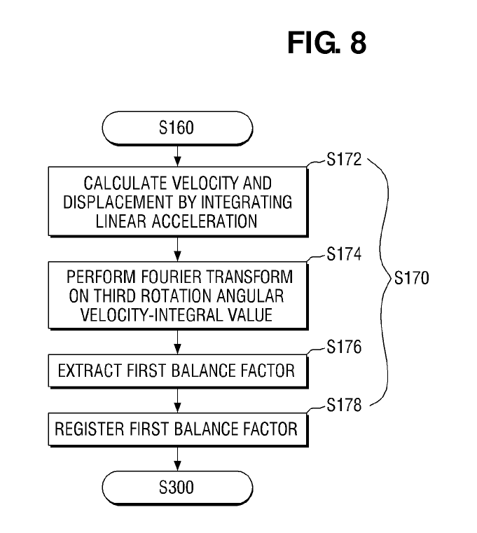

In detail, referring to FIGS. 2 and 8, the control unit 130 can calculate velocity and displacement by integrating the linear acceleration (S172), perform Fourier transform on the third rotation angular velocity-integral value (S174), and extract the first balance factor on the basis of the velocity, the displacement, and the Fourier transformed-third rotation angular velocity-integral value (S178). Further, the control unit 130 can register the extracted first balance factor (S178) and store it in the memory 120.

Referring to FIGS. 2 and 4 again, after the first balance factor of any one (for example, the right arm) of the user's left and right arms is registered (S100), the motion of the other one (for example, the left arm) of the user's left and right arms is detected (S300).

In detail, after the first balance factor of any one (for example, the right arm) of the user's left and right arms is registered (S100), the motion sensor 110 is activated periodically or by the controlling of the control unit 130 and motion data can be created by detecting the motion of the other one (for example, the left arm) of the user's left and right arms for a predetermined time with the motion sensor 110. For example, when the motion sensor 110 is an acceleration sensor, the smart band 100 measures the acceleration of a user's motion and creates acceleration data, and when the motion sensor 110 is a gyroscope, it measures a rotation angular velocity of a user's motion and creates angular velocity data. The acceleration data contains three axial (x-, y-, and z-axial) acceleration components and the angular velocity data contains three axial angular velocity components.

Next, the second balance factor of the other one (for example, the left arm) of the user's left and right arms is extracted (S400).

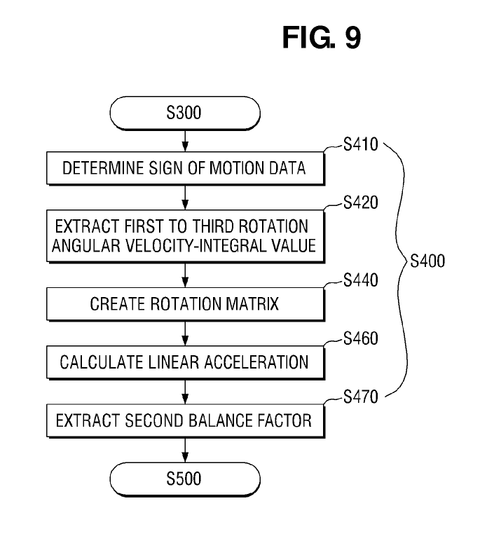

In detail, referring to FIGS. 2 and 9, the sign of the motion data is determined first (S410).

In detail, the control unit 130 can determine the sign of the motion data by checking whether the user's motion is the motion of the user's left arm or right arm.

Next, the first to third rotation angular velocity-integral values are extracted (S420).

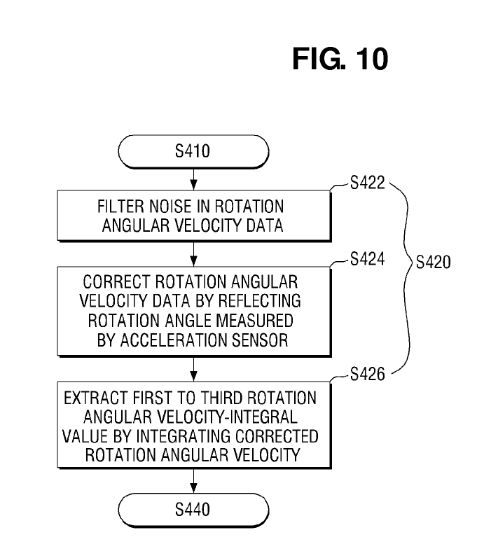

In detail, referring to FIGS. 2 and 10, the control unit 130 can first filter noises in the rotation angular velocity data of the motion data with a sign determined (in which, the filtering may be achieved by a filter (not illustrated) in the control unit 130) (S422), correct the rotation angular velocity data with noises filtered, by reflecting the rotation angle measured by the acceleration sensor (not illustrated) (S424), and extract the first to third rotation angular velocity-integral values by integrating the corrected rotation angular velocity (S426).

Referring to FIGS. 2 and 9 again, a rotation matrix is created (S440).

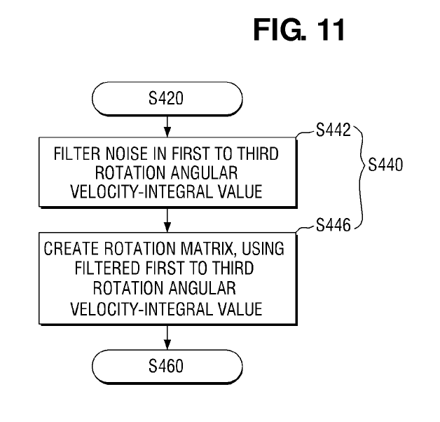

In detail, referring to FIGS. 2 and 11, the control unit 130 can filter noises in the extracted first to third rotation angular velocity-integral values (in which, the filtering may be achieved by a filter (not illustrated) in the control unit 130) (S442) and create a rotation matrix, using the filtered first to third rotation angular velocity-integral values (S446).

Referring to FIGS. 2 and 9 again, linear acceleration is calculated (S460).

In detail, the control unit 130 can calculate the linear acceleration by applying the rotation matrix to the acceleration data of the motion data measured by the motion sensor 110.

Next, the second balance factor is extracted (S470).

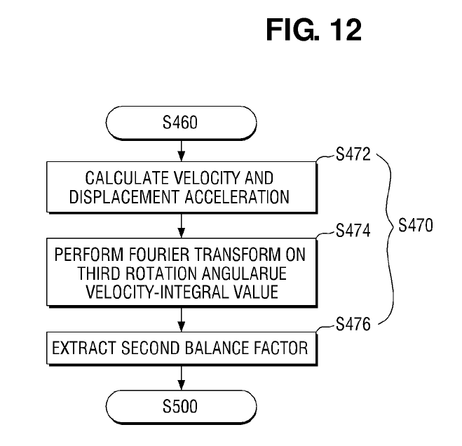

In detail, referring to FIGS. 2 and 12, the control unit 130 can calculate velocity and displacement by integrating the linear acceleration (S472), perform Fourier transform on the third rotation angular velocity-integral value (S474), and extract the second balance factor on the basis of the velocity, the displacement, and the Fourier transformed-third rotation angular velocity-integral value (S478).

Referring to FIGS. 2 and 4 again, after extracting the second balance factor (S478), an asymmetry index is calculated (S500).

In detail, the control unit 130 can calculate the asymmetry index on the basis of the difference between the second balance factor and the first balance factor stored in the memory 120.

Next, the final score is calculated (S600).

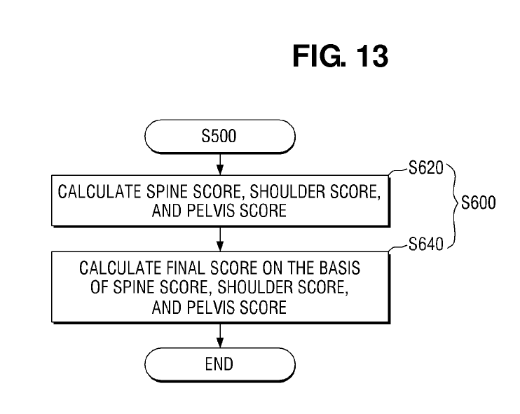

In detail, referring to FIGS. 2 and 13, the control unit 130 can calculate a spine score, a shoulder score, and a pelvis score on the basis of the asymmetry index (S620) and can calculate the final score on the basis of the spine score, the shoulder score, and the pelvis score.

The final score calculated through this algorithm can be displayed on the display unit 160 and the user can find out which state his/her body balance was in from the final score.

For example, the higher the final score, the better the body balance, while the lower the final score, the worse the body balance, but the present invention is not limited thereto.

Thereafter, the smart band 100 ends the algorithm according to an embodiment of the present invention.

The method of measuring body balance of a smart band according to embodiments of the present invention described above can be achieved as a computer-readable code or program on a computer-readable recording medium. The computer-readable recording medium includes all kinds of recording media storing data readably by a computer system. That is, the computer-readable media may include program commands, data files, and data structures, or combinations thereof. The program command that are recorded on the recording media may be those specifically designed and configure for the present invention or may be those available and known those engaged in computer software in the art. The computer-readable recording medium may be ROM, RAM, CD-ROM, magnetic tape, floppy disc, and optical data storage, and may be implemented in a carrier wave type (for example, transmitted by internet). The computer-readable recording medium may be distributed to a computer system that is connected through a network and may store and execute computer-readable codes in the type of distribution.

The foregoing is illustrative of the present invention and is not to be construed as limiting thereof. Although a few embodiments of the present invention have been described, those skilled in the art will readily appreciate that many modifications are possible in the embodiments without materially departing from the novel teachings and advantages of the present invention. Accordingly, all such modifications are intended to be included within the scope of the present invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The present invention is defined by the following claims, with equivalents of the claims to be included therein.

What is claimed is:

1. A smart band comprising:
a memory that stores a first balance factor of any one of user's left and right arms;
a motion sensor that creates motion data by detecting a motion of the other one of the user's left and right arms; and
a control unit that extracts a second balance factor of the other one of the user's left and right arms on the basis of the created motion data, calculates an asymmetry index, using the first and second balance factors, and calculates a final score on the basis of the asymmetry index.

2. The smart band of claim 1, wherein the motion data contains acceleration or rotation angular velocity of the user's motion.

3. The smart band of claim 1, wherein the control unit determines a sign of the motion data by checking whether the user's motion is the motion of the user's left arm or right arm.

4. The smart band of claim 3, wherein the sign of motion data for a motion of the user's left arm and the sign of motion data for a motion of the user's right arm are opposite to each other.

5. The smart band of claim 1, wherein the motion sensor includes an acceleration sensor measuring acceleration of a user's motion and a gyroscope measuring a rotation angular velocity of a user's motion.

6. The smart band of claim 5, wherein the control unit corrects the rotation angular velocity measured by the gyroscope by reflecting a rotation angle measured by the acceleration sensor, extracts first to third rotation angular velocity-integral values by integrating the corrected rotation angular velocity, filters noises in the extracted first to third rotation angular velocity-integral values, and creates a rotation matrix, using the filtered first to third rotation angular velocity-integral values.

7. The smart band of claim 6, further comprising:
a filter for filtering noises in the first to third rotation angular velocity-integral values,
wherein the filter filters noises in the rotation angular velocity measured by the gyroscope before the correcting.

8. The smart band of claim 6, wherein the control unit calculates linear acceleration by applying the rotation matrix to the acceleration measured by the acceleration sensor, calculates velocity and displacement by integrating the linear acceleration, performs Fourier transform on the third rotation angular velocity-integral value, and extracts the second balance factor on the basis of the velocity, the displacement, and the Fourier transformed-third rotation angular velocity-integral value.

9. The smart band of claim 8, further comprising:
a display unit that displays the final score,
wherein directional axes of each of the first and second rotation angular velocity-integral values cross each other and are positioned in the same plane as a liquid crystal surface of the display unit, and
a directional axis of the third rotation angular velocity-integral value crosses the directional axes of each of the first and second rotation angular velocity-integral values and is perpendicular to the liquid crystal surface of the display unit.

10. The smart band of claim 1, wherein the control unit receives the first balance factor from the memory and calculates the asymmetry index on the basis of the difference between the first balance factor and the second balance factor, and
each of the first and second balance factors contain a plurality of sub-balance factors.

11. The smart band of claim 1, wherein the control unit calculates a spine score, a shoulder score, and a pelvis score on the basis of the asymmetry index and calculates the final score on the basis of the spine score, the shoulder score, and the pelvis score.

12. A method of measuring body balance of a smart band, the method comprising:
creating motion data by detecting a motion of any one of user's left and right arms;
extracting a first balance factor of any one of the user's left and right arms on the basis of the motion data;
calculating an asymmetry index, using the first balance factor and a second balance factor of the other one of the user's left and right arms stored in a memory; and
calculating a final score on the basis of the asymmetry index.

13. The method of claim 12, wherein the extracting of the first balance factor includes:
determining a sign of the motion data by checking whether the user's motion is the motion of the user's left arm or right arm;
extracting first to third rotation angular velocity-integral values on the basis of rotation angular velocity data of the motion data with the determined sign;
creating a rotation matrix on the basis of the extracted first to third rotation angular velocity-integral values;
calculating linear acceleration by applying the rotation matrix to acceleration data of the measured motion data; and
extracting the first balance factor on the basis of the linear acceleration and the third rotation angular velocity-integral value.

14. The method of claim 13, wherein the extracting of first to third rotation angular velocity-integral values on the basis of rotation angular velocity data of the motion data with the determined sign includes:
correcting rotation angular velocity data of the motion data with the determined sign, by reflecting a rotation angle measured by an acceleration sensor; and
extracting first to third rotation angular velocity-integral values by integrating the corrected rotation angular velocity data.

15. The method of claim 14, further comprising:
filtering noises in the rotation angular velocity data, before the correcting of rotation angular velocity data by reflecting a rotation angle measured by an acceleration sensor.

16. The method of claim 13, wherein the creating a rotation matrix on the basis of the extracted first to third rotation angular velocity-integral values includes:
filtering noises in the extracted first to third rotation angular velocity-integral values; and
creating a rotation matrix, using the filtered first to third rotation angular velocity-integral values.

17. The method of claim 13, wherein the extracting of the first balance factor on the basis of the linear acceleration and the third rotation angular velocity-integral values includes:
calculating velocity and displacement by integrating the linear acceleration;
performing Fourier transform on the third rotation angular velocity-integral value; and
extracting the second balance factor on the basis of the velocity, the displacement, and the Fourier transformed-third rotation angular velocity-integral value.

18. The method of claim 12, wherein the calculating of an asymmetry index includes:
calculating the asymmetry index on the basis of the difference between the first balance factor and the second balance factor.

19. The method of claim 18, further comprising registering the second balance factor of the other one of the user's left and right arms which is compared with the first balance factor, before the creating of motion data by measuring a motion of any one of the user's left and right arms.

20. The method of claim 12, wherein the calculating of a final score on the basis of the asymmetry index includes:
calculating a spine score, a shoulder score, and a pelvis score on the basis of the asymmetry index; and
calculating the final score on the basis of the spine score, the shoulder score, and the pelvis score.
